(19) United States
(12) Patent Application Publication
(10) Pub. No.: US 2026/0345049 A1
(43) Pub. Date: Nov. 5, 2026

(54) IMPLANTABLE HIGHLY FLEXIBLE BRAIDED MULTI-ELECTRODE PROBES (BMEPS) FOR ELECTRICAL STIMULATION AND ELETROMYOGRAPHY RECORDING IN MUSCLES

(71) Applicant: Drexel University, Philadelphia, PA (US)

(72) Inventors: Simon F. Giszter, Havertown, PA (US); Tae Gyo Kim, Conshohocken, PA (US); Benjamin Binder-Markey, Jenkintown, PA (US)

(21) Appl. No.: 19/105,107

(22) PCT Filed: Aug. 24, 2023
(86) PCT No.: PCT/US23/31062

§ 371 Date: Feb. 20, 2025

Related U.S. Application Data
(60) Provisional application No. 63/400,840, filed on Aug. 25, 2022.

(51) Int. Cl. A61B 5/296; A61N 1/05

(52) U.S. Cl. CPC A61B 5/296; A61N 1/0558; A61B 2562/043; A61B 2562/125; A61B 2562/164

(57) ABSTRACT

An implantable probe is provided herein. The implantable probe includes a plurality of wires configured for at least one of electromyography and electrical stimulation and a securing element formed at an end of the plurality of wires, wherein the plurality of wires are in a braided configuration and are configured to transmit multiple electrical signals in connection with a biological material. Also provided herein are a syringe based implantable probe system for positioning the implantable probe, and a method of making the implantable probe.

### CROSS-REFERENCE TO RELATED APPLICATION(S)

The present application claims priority under 35 U.S.C. § 119(e) to U.S. Provisional Patent Application No. 63/400,840, filed Aug. 25, 2022, which application is incorporated herein by reference in its entirety.

### STATEMENT REGARDING FEDERALLY SPONSORED RESEARCH OR DEVELOPMENT

This invention was made with government support under grant numbers NINDS RO1 NS072651 and NIBIB U01 EB021921 awarded by the National Institute of Health (NIH) and grant number IIS 1515140 awarded by the National Science Foundation (NSF). The government has certain rights in the invention.

### BACKGROUND

Critical diagnostic data for neural and neuromuscular disorders are collected from intramuscular electromyography (EMG). Currently, electrodiagnostic EMG is as much an art as science, requiring extremely skilled individuals for the reliable acquisition and interpretation of results, which can vary from tester to tester. Additionally, electrodiagnostic EMG is currently performed with stiff needles, which do not permit the acquisition of EMG in conditions allowing free movement.

Electrical stimulation (E-Stim) of a muscle is commonly used treatments for therapeutic maintenance of muscle, to reawaken awareness of muscles, and to manage pain. Such stimulations are usually performed by physical medicine and rehabilitation (PM&R) doctors and physical therapists (PTs) in a hospital setup. In some instances, when uncomplicated surface stimulation is possible, out of clinic take home units may be used. However, this is not possible for deep muscles, such as multifidus in lower back pain. Instead, the current E-Stim in multifidus or other deep muscles is performed only with a surface electrode pad laid on skin. When using a surface electrode, electrical current must pass through the skin and multiple subcutaneous layers to reach the muscle, and is spread in all directions from the electrode pad, losing selectivity and causing spurious activations and pain. This results in the applied electrical current quickly dissipating via multiple barriers and very broadly affects the target muscle. In other words, the efficiency and effectiveness of E-Stim with the surface electrode on a muscle is very low.

Furthermore, for the E-Stim of the deeper part of a muscle, higher level of electrical current is required, which can cause unwanted additional electrical pain. To overcome these problems, intramuscular E-Stim in-clinic with an injectable electrode such as a metal needle has been proposed as an alternative. However, intramuscular E-Stim is currently not available in PM&R centers at all sites, and never at home, and even dry needling with E-Stim by PTs has been very rarely performed for research purposes only.

It would be desirable to provide a system that simplifies the current procedures to allow consistent and rapid acquisition of EMG data and improved E-Stim.

### SUMMARY

In one aspect, provided herein is an implantable probe. The implantable probe includes a plurality of wires configured for at least one of electromyography and electrical stimulation, and a securing element formed at an end of the plurality of wires, wherein the plurality of wires are in a braided configuration and are configured to transmit multiple electrical signals in connection with a biological material. In some embodiments, the probe further includes a removable core portion. In some embodiments, the plurality of wires are configured to be braided around an exterior surface of the core portion.

In some embodiments, the securing element is configured to maintain a position of the plurality of wires with respect to the core portion during implantation of the probe. In some embodiments, the securing element is configured to release from the core portion when the core portion is withdrawn from an implantation site. In some embodiments, the securing element includes a ring or cap. In some embodiments, the securing element comprises a polyimide material.

In some embodiments, the plurality of wires are highly flexible multichannel microelectrodes. In some embodiments, the plurality of wires are tubularly braided microwires. In some embodiments, the plurality of wires are configured to release from a core portion after insertion of the implantable probe into a biological material. In some embodiments, the plurality of wires include a tapered end configured to release from a core portion upon withdrawal of the core portion from the biological material.

In another aspect, provided herein is a syringe-based implantable probe system including a syringe, a core portion attached to a plunger within the syringe; and the implantable probe according to one or more of the embodiments disclosed herein arranged and disposed to be positioned over the core portion. In some embodiments, the core portion includes a PTFE or polyimide coating.

In another aspect, provided herein is a method of making the implantable probe, the method comprising (a) tubularly braiding the plurality of wires on a core portion; (b) making electrical connections between the braided plurality of wires on the core portion and respective channels of a multichannel connector; (c) applying a coating on a portion of the braided microwires; and (d) cutting the braided plurality of wires within the coating to form a coating tapered ring structure. In some embodiments, the method further includes the step of (e) applying another polyimide coating at the tips of the braided plurality of wires exposed by the cutting of step (d). In some embodiments, the method further includes the step of (f) disposing a plurality of interaction sites on the braided plurality of wires via an ablation process.

In some embodiments, the braided plurality of wires are in a single 12 filament tubular braid configuration. In some embodiments, the braided plurality of wires are in a double 12 filament tubular braid configuration.

In some embodiments, during step (b) the connector is attached to the syringe body by at least one of a mechanical holder and a magnetic holder.

In some embodiments, the coating is applied over only the portion of the braided microwires corresponding to a distal end of the core portion.

In some embodiments, the coating tapered ring portion is configured to maintain a position of the tubularly braided plurality of wires with respect to the core portion during insertion of the probe into a muscle. In some embodiments, the coating tapered ring structure is further configured to release from the core portion when the core portion is withdrawn from the muscle.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 illustrates a side view of an implantable probe, in accordance with an exemplary embodiment of the present disclosure.

FIGS. 2A-2G illustrate various views of an implantable probe. (A) illustrates a side view of an implantable probe around a needle and the interaction sites disposed thereon, in accordance with an exemplary embodiment of the present disclosure. (B-G) illustrate various portions of interaction sites disposed on the implantable probe of (A), in accordance with an exemplary embodiment of the present disclosure.

FIG. 3 shows a photograph of an implantable probe, in accordance with an exemplary embodiment of the present disclosure.

FIGS. 4A-4C show photographs illustrating insertion of an implantable probe into a biological material and removal of the core portion, in accordance with an exemplary embodiment of the present disclosure. (A) Insertion of the implantable probe and core portion. (B) Substantial removal of the core portion while leaving the implantable probe in place. (C) Enlarged view of the implantable probe and core portion during removal of the core portion.

FIGS. 5A-5D illustrate a block diagram side view of insertion of an implantable probe into a biological material and removal of a core portion therefrom, in accordance with an exemplary embodiment of the present disclosure. (A) Insertion of the implantable probe and core portion. (B-C) Removal of the core portion from the inserted probe. (D) Inserted probe following removal of the core portion.

FIGS. 6A-C illustrate a probe delivery system. (A) Perspective view of a probe delivery system. (B) Enlarged view of inset B showing a distal end of the probe attached to the delivery system. (C) Enlarged view of inset C showing a proximal end of the probe attached to the delivery system, where the probe meets a blunt needle dispenser on a tip of the syringe.

FIGS. 7A-7B illustrate the probe delivery system in a deployed state (A) and a withdrawn state (B).

FIG. 8 shows photographs illustrating a multichannel connector in a holder mounted on a syringe.

FIG. 9 shows a photograph illustrating EMG recording in a rat using a fully assembled deployment and connection mechanism.

FIGS. 10A-B illustrate example single motor unit (SMU) data from deployed flexible BMEP in a rat. (A) Filtered raw EMG with 8-channel BMEP EMG needle. (B) Sorted SMUs across all channels.

FIGS. 11A-B illustrate expanded time scale raw data from rat with flexible BMEP deployed. (A) Filtered raw EMG with 8-channel BMEP EMG needle. (B) Enlarged view of timescale identified in (A). Separated units are color coded.

FIGS. 12A-B illustrate unit separation in PCA based single unit clustering in rat data. (A) Sorted 3D PCA in all channels from PC1 and PC2. (B) Sorted 3D PCA in all channels from PC1, PC2, and PC3.

### DETAILED DESCRIPTION OF THE INVENTION

Provided herein is a novel probe of highly flexible multichannel microelectrodes. The probe disclosed herein may be injected and/or implanted (e.g., into a muscle) for acute, semi-chronic, or chronic electrical muscle stimulation and/or EMG (Electromyograph) recording.

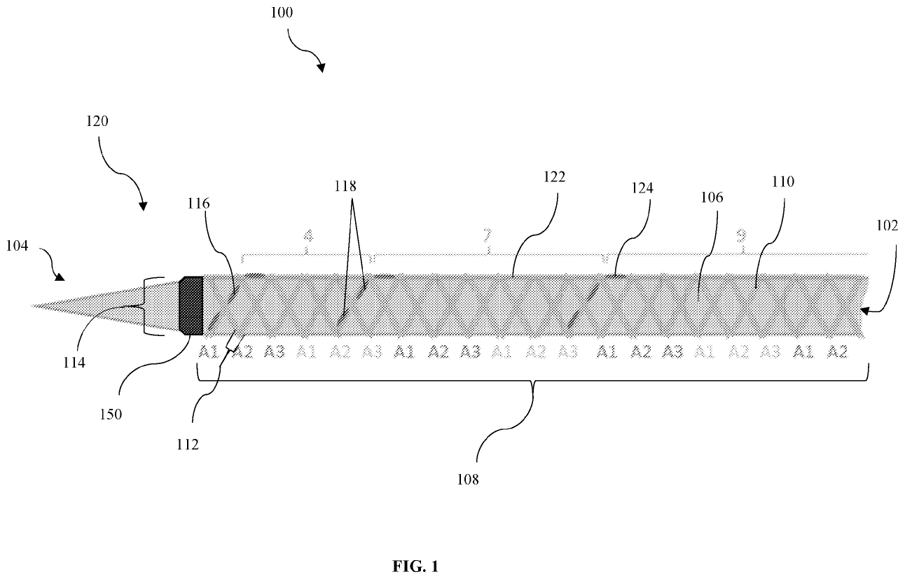

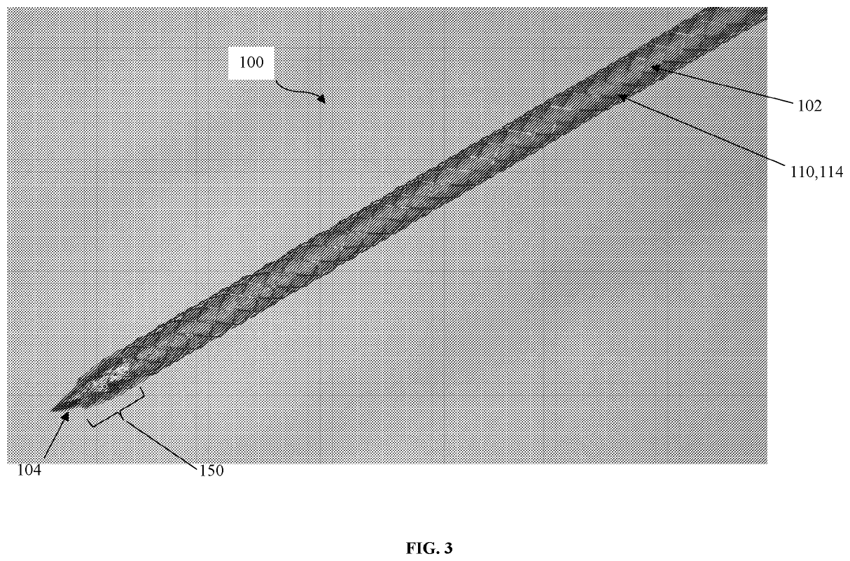

Referring to FIGS. 1-3, in some embodiments, the probe 100 includes a plurality of wires 110 configured for electromyography. In some embodiments, the plurality of wires 110 is positioned over and/or around a core portion 102. Additionally or alternatively, in some embodiments, the probe 100 includes a securing element 150 connected to an end of the plurality of wires 110.

The core portion 102 includes an elongated portion 108 with a tip portion 104 at a distal end 120 thereof. The tip portion 104 may include the same shape as the rest of the core portion 102, a blunt end, a tapered end, or any other suitable shape for positioning within and removal from the plurality of wires 110. In some embodiments, the tip portion 104 of the core portion 102 is arranged and disposed to facilitate insertion of the probe 100 into biological tissue (e.g., muscle). For example, in some embodiments, the core portion 102 includes a needle (e.g., microneedle) with a tapered tip portion 104. Although described herein primarily with respect to a needle, the core portion 102 is not so limited and may include any suitable shape and/or material for supporting the plurality of wires 110 and/or facilitating injection/implantation thereof.

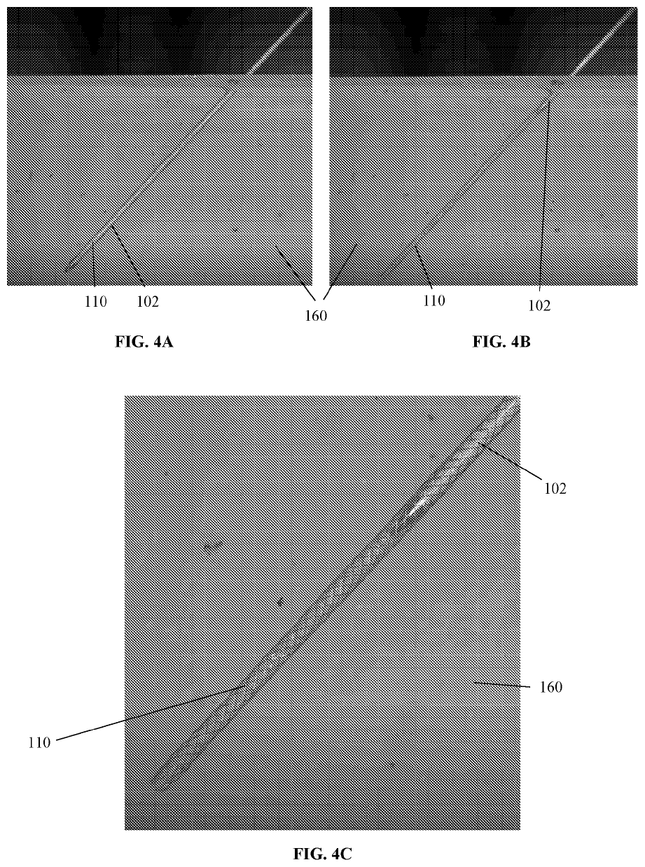

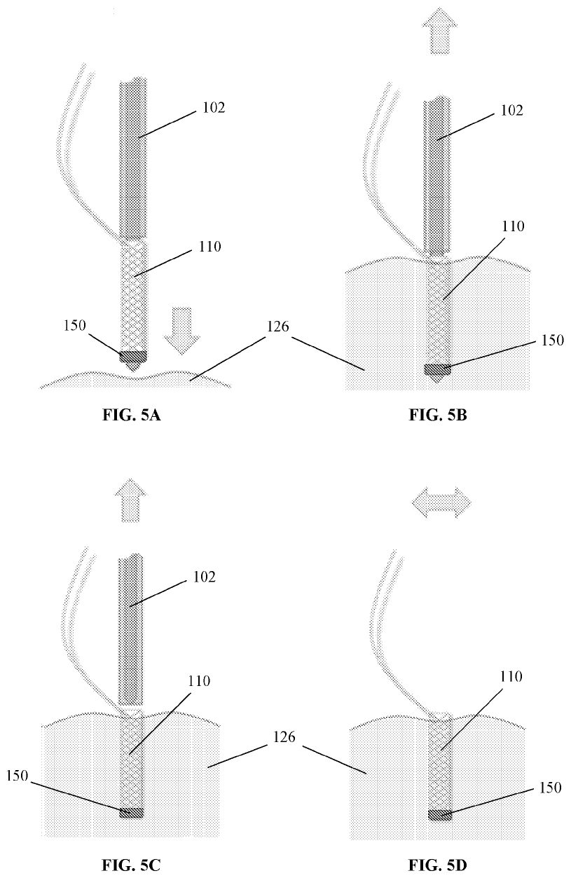

In some embodiments, the core portion 102 acts as a mandrel during the formation of the plurality of wires 110 (e.g., tubularly braided microwires). Additionally or alternatively, as illustrated in FIGS. 4A-5D, the core portion 102 may be removable from the plurality of wires 110 (e.g., a microneedle removable from within the lumen of a tubular braid). In some embodiments, the core portion 102 (e.g., needle) is rigid, or at least comparatively more rigid than the plurality of wires 110, in order to provide support during and/or facilitate the positioning of the plurality of wires 110. Suitable materials for the core portion 102 include, but are not limited to, tungsten, stainless steel, or other stiff metals, other non-toxic and biocompatible materials, carbon, or a combination thereof. In some embodiments, the core portion 102 is at least partially coated with a material, such as, but not limited to, a material with a low friction, an adhesive material, a non-toxic material, and/or biocompatible material. Suitable coating materials include, but are not limited to, PTFE and/or silicone. After positioning, the core portion 102 may be removed (e.g., withdrawn from the biological material), leaving the plurality of wires 110 at the injection/implantation site without the core portion 102. Following removal of the core portion 102, the plurality of wires 110 (e.g., braided tubule) are free to flex, extend, and/or compress as needed during movement at the injection/implantation site.

In some embodiments, the plurality of wires 110 include microwires. In some embodiments, the plurality of wires 110 are braided 114. For example, in some embodiments, the plurality of wires 110 are arranged in a tubularly braided configuration 114 around an exterior surface 106 of the core portion 102. In some embodiments, the braid configuration includes a single 12 filament tubular braid configuration. In some embodiments, the braid configuration includes a double 12 filament tubular braid configuration. Although described herein primarily with respect to tubular braided microwires, as will be appreciated by those skilled in the art, the disclosure is not so limited and the plurality of wires 110 may include any suitable shape, material, and/or arrangement for injection/implantation and use as described herein. Additionally or alternatively, the plurality of wires 110 may be grouped into groupings 112 (e.g., two wires, three wires, four wires, five wires, six wires, seven wires, eight wires, etc.). In some embodiments, the plurality of wires 110 (and/or the groupings 112) may be arranged in a helical pattern (illustrated in FIG. 1 around the core portion 102). In some embodiments, the plurality of wires 110 (and/or the groupings 112) are interleaved with (or wrapped around) a lay-in structure 122, such as a lay-in wire, running along the length of core portion 102 and/or elongated portion 108. When positioned over and/or around the core portion 102, the plurality of wires 110 terminate at the distal end 120 thereof.

The plurality of wires 110 can be made of a material suitable for use in connection with braiding and/or electromyography. The plurality of wires 110 can be described as highly flexible. The braid design of the plurality of wires 110 leverages the change in bending stiffness achieved using cylindrical elements with their change in radius, a relationship which is fourth order. For example, Kim et al. 2013 have demonstrated a 20 fold reduction in bending stiffness (i.e., 20× increase in compliance or flexibility) of a tubular helical braid of 12 9.6 micron wires, relative to a single 50 micron wire, despite only ~30% reduction in material in the braid as compared to a single wire.

In certain embodiments, the plurality of wires 110 can be made from a biocompatible or non-toxic alloy. In certain embodiments, the plurality of wires 110 electrically conductive material in a fully hermetic encapsulation. In certain embodiments, plurality of wires 110 can be made (at least in part) from nichrome, steel, platinum, platinum iridium, gold, or other suitable materials. In certain application, a coating (e.g., polyimide, a biocompatible electrical insulation material suited to braiding and ablation, etc.) may be applied to the plurality of wires 110.

In some embodiments, the probe 100 is configured to transmit multiple electrical signals in connection with a biological material. The electrical signal of a biological material may be generated in a muscle associated with motor activity or pathological motor behavior (or resulting from the delivery of electrical current). The electrical signal of the biological material may be motor unit potentials, single motor units, single fiber potentials, electrical voltage or signals needed to estimate electrical impedance (e.g., a current-controlled or voltage-controlled sinewave function input may be used to measure signals used to derive impedance, resistance, inductance, capacitance, etc.). For example, in some embodiments, the probe 100 includes a braided multi-electrode probes (BMEPs) for electrical stimulation and electromyography recording in muscles.

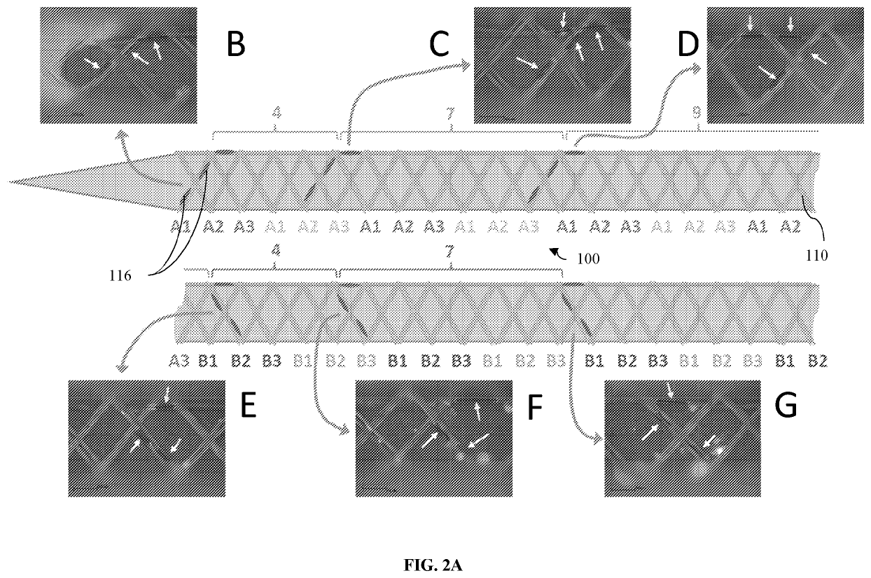

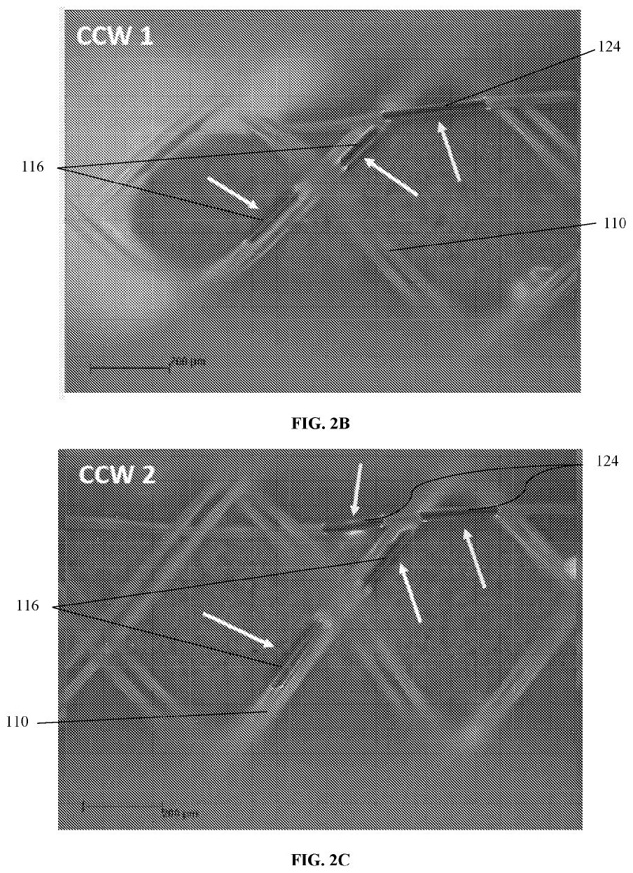

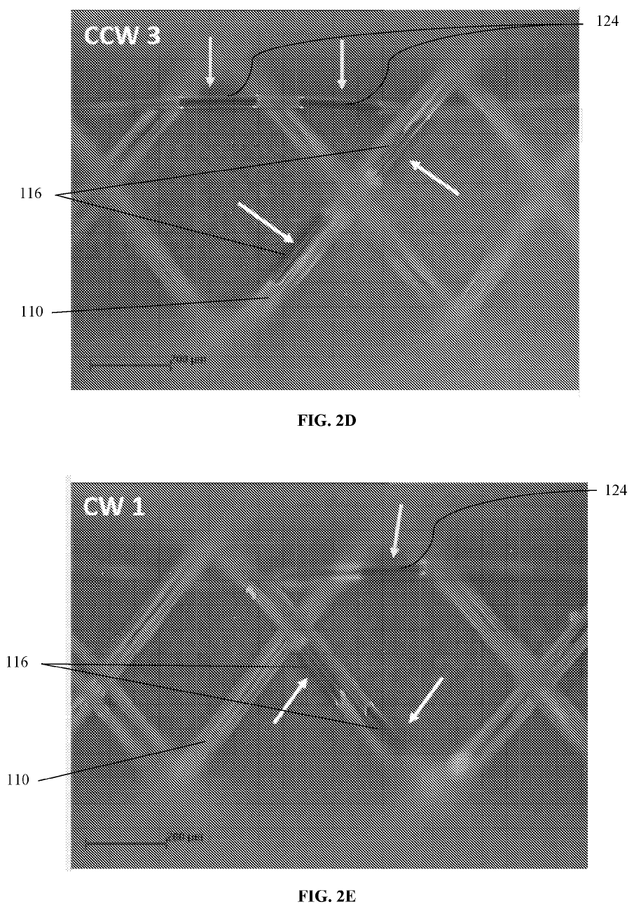

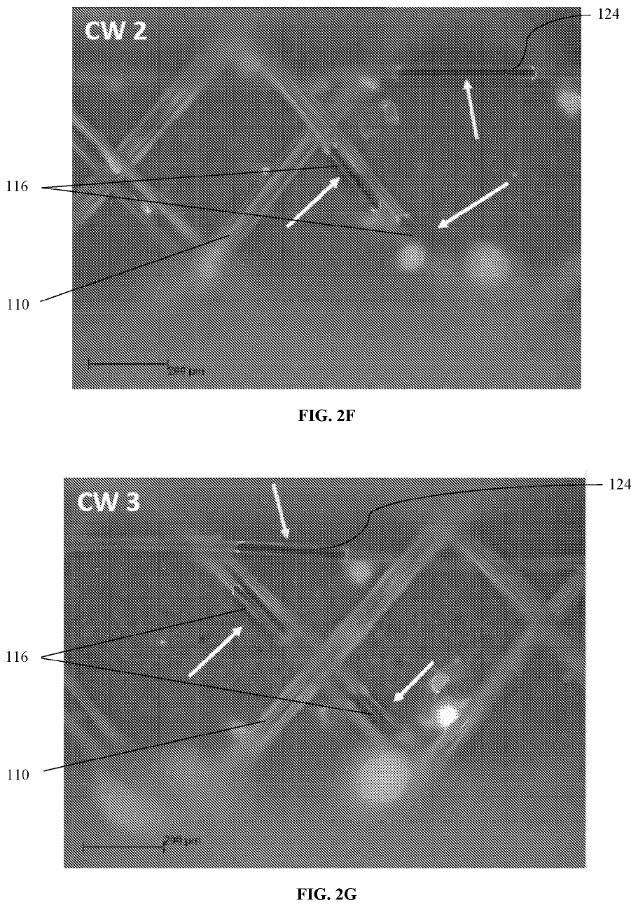

Referring to FIGS. 2A-2G, in some embodiments, the plurality of wires 110 and/or the lay-in structure 122 include at least one interaction site (e.g., recording site, communication site, transmission site, etc.). In some embodiments, interaction sites 116 (and/or interaction sites 118) are disposed on one or more of the plurality of wires 110. For example, interaction sites 116 can be disposed on certain portions of certain wires 110 in groupings (e.g., grouping 112). Additionally or alternatively, in some embodiments, interaction sites 124 (e.g., reference interaction sites, reference recording sites, etc.) are disposed along the along lay-in structure 122.

These interaction sites may be configured to measure an electrical signal from biological material (e.g., muscle tissue). For example, the interaction sites of implantable probe 100 can be brought into contact with biological material at various locations. The relationship (e.g., spatial relationship) between each of interaction sites 116 and interaction sites 124 can then be used to perform multichannel analyses. In this regard, the size and placement (i.e., absolute placement or relative placement) of the interaction sites may be varied depending on the desired measurement (e.g., using multichannel analysis, signal triangulation, etc.) or desired use of the interaction sites (e.g., measuring electrical signals, transmitting electrical signals in a stimulation application, etc.). For example, in some embodiments, the sensing and/or stimulating sites may be arranged as disclosed in U.S. Pat. Nos. 8,639,311 and/or 11,504,524, which are incorporated herein by reference in their entirety. The interaction sites can be disposed on wires 110 and/or the lay-in structure 122 at certain locations using an ablation process.

In some embodiments, the securing element 150 includes one or more features to retain and/or secure the position of the plurality of wires 110 with respect to the removable core portion 102. For example, the plurality of wires 110 (e.g., tubular braid) may narrow over a tapered tip portion 104 of the core portion 102 (e.g., the needle tip). Additionally or alternatively, the securing element 150 may be coupled to the plurality of wires 110, the securing element 150 having an opening (e.g., a ring of material) or recessed portion (e.g., a bullet shaped material) that engages the tip portion 104 of the core portion 102 without being adhered thereto. The securing element 150 may be formed from any suitable material for coupling to the plurality of wires 110 and engaging the tip portion 104 of the core 102, such as, but not limited to, polyimide. In such embodiments, the narrowing of the plurality of wires 110 and/or the securing element 150 prevents the plurality of wires 110 from sliding along the core portion 102 during positioning of the probe 100 (e.g., insertion into a biological material/tissue).

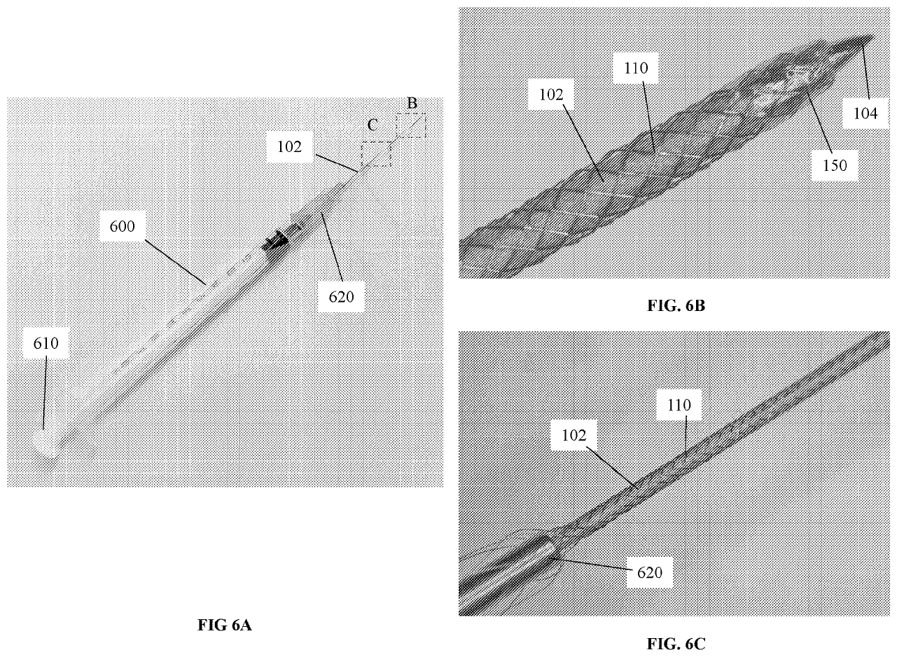

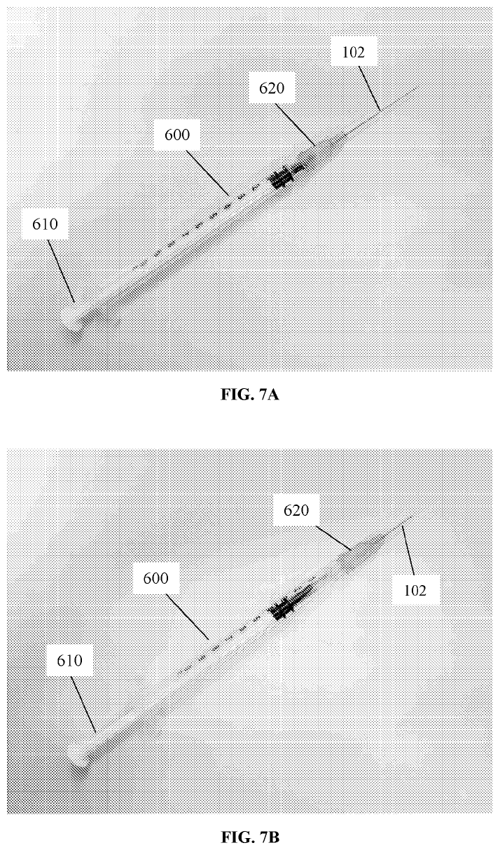

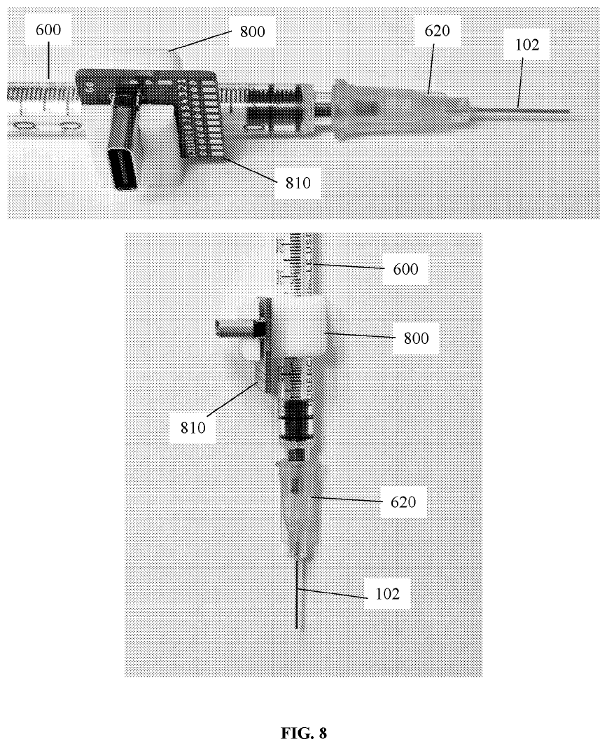

Also provided herein are articles and methods for positioning the probe 100. Referring to FIGS. 6A-7B, in some embodiments, the probe is positioned using a syringe 600. For example, in some embodiments, the core portion is attached to the plunger/piston 610 in the syringe 600 (e.g., 1 ml size), such that pulling and pushing the piston 610 moves the core portion 102 in and out of the end of the syringe 600. In such embodiments, when the plunger 610 is depressed and the core portion 102 is in a fully extended/deployed state (FIG. 7A), the plurality of wires 110 may be positioned over the core portion 102 and the probe 100 may be injected/implanted (e.g., into a muscle). After injecting/implanting the probe 100, the piston 610 may be pulled to withdraw the core portion 102 (FIG. 7B) and leave only the probe 100 in the muscle. In some embodiments, the core portion 102 is completely hidden inside the syringe 600 when the piston 610 is fully withdrawn.

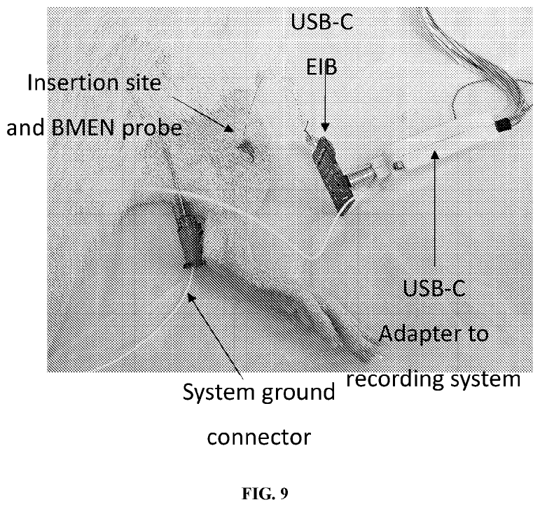

In some embodiments, the tip of the syringe 600 permits the core portion 102 to slide through, but prevents the plurality of wires 110 from retracting therewith (i.e., the tip of the syringe is wider than a diameter of the probe). Additionally or alternatively, in some embodiments, the syringe includes a blunt needle dispenser 620 having a larger diameter to further ensure that the probe/plurality of wires 110 does not retract with the core portion 102 when the plunger 610 is pulled. Accordingly, after inserting the probe 100 and core portion 102 into a tissue, the tip of the syringe 600 and/or the blunt needle dispense 620 positioned on the tip prevent the probe 100 from retracting with the core portion 102, ensuring that the probe 100 remains within the injection/implantation site (FIG. 9).

Turning to FIG. 8, in some embodiments, a connection 810 for the plurality of wires 110 is coupled to the syringe 600. For example, in some embodiments, a USB-C EIB connector 810 designed for BMEN is positioned in a slotted holder 800 mounted on the syringe 600. In such embodiments, when the plurality of wires 110 is coupled to the USB-C EIB connector 810 before the probe 100 is implanted, the slotted holder 800 mounted on the syringe 600 maintains the position of the connector 810 relative to the probe during insertion.

Further provided herein are methods of manufacturing implantable probes (e.g., BMEP for muscle applications). In some embodiments, the method includes two processes: making the syringe-based removable needle system and making an implantable probe (e.g., BMEP) on the syringe-based removable needle system.

### Syringe-Based Removable Needle System

Certain methods of making a syringe-based removable needle system include the steps of: (i) attaching a metal rod to a syringe set; (ii) sharpening a tip of the metal rod; and (iii) optionally coating the sharpened tip with a material (e.g., PTFE).

In step (i), a metal rod is prepared in a length. Materials for the rod could be any kind of stiff metal, such as tungsten, stainless steel, etc. The rod can be made from certain stiff and robust materials suitable for making a penetrating needle. In certain embodiments, the rod can be made from certain non-toxic and biocompatible materials (e.g., with regard to the duration the needle is in contact with biological material). In certain embodiments, the rod can be made (at least in part) from carbon (e.g., with suitable form and structural material composition). In certain applications, the rod can be coated with a material with a low friction, adhesive material, non-toxic, and/or biocompatible material. In certain applications, the rod can be coated with (at least in part) PTFE, silicone, or a similar composition.

The diameter and length of the rod are also customizable depending on applications. In one example, the rod can be a 254 μm diameter tungsten rods with a one (1) inch length. A first end of a metal rod is perpendicularly inserted into a top surface of piston rubber via a tunnel of a syringe body (e.g., a 1 ml syringe body). A second end of the metal rod is inserted into a blunt tip syringe dispenser. The dispenser is attached to the syringe body for full assembly.

In step (ii), the tip of rod extruded from the blunt tip syringe needle is sharpened. This sharpening process can involve grinding (grounding) with sandpaper, chemical etching, or another process. Alternatively, a rod with a pre-sharpened tip may be used, thus avoiding the sharpening step.

Depending upon the material of the rod, in step (iii), the sharpened tip of rod is optionally coated (e.g., by PTFE) to decrease surface friction of the sharp portion of needle. This coating may facilitate movement of the rod with respect to the plurality of wires and/or a securing element coupled thereto (e.g., a polyimide coating layer which is described in making implantable probe).

### Making the Implantable Probe

Manufacturing or making an implantable probe (e.g., a BMEP on a syringe-based removable needle system) can include the steps of: (a) tubularly braiding the plurality of wires on a needle system; (b) making electrical connections between ones of the braided plurality of wires on the needle system and respective channels of a multichannel connector; (c) applying a polyimide coating on braided microwires over only a tapered/sharpened end of the core portion (e.g., the pointed end of a needle); and (d) cutting the braided plurality of wires within the polyimide coating to form a polyimide coating tapered ring structure configured to enable the braided plurality of wires to be inserted into a muscle, the polyimide coating tapered ring structure being further configured to be released smoothly from the needle of the needle system when the needle is removed.

In step (a), a plurality of wires (e.g., 12-24 microwires) are tubularly braided on the needle system prepared as described above. The plurality of wires may be braided through any suitable method, such as, but not limited to, with a custom microbraiding machine. For example, in some embodiments, the plurality of wires are braided according to the method described in U.S. Pat. No. 8,534,176, which is incorporated herein by reference in its entirety. The configuration can be of single or double 12 filaments tubular braid.

In step (b) electrical connections are made between individual wires of the plurality of wires (e.g., braided microwires on the needle system) and a connector (e.g., a 12-channel connector). In certain embodiments, the connector could be attached to the syringe body via a mechanical holder or magnetic holder. The holder designs vary depending on types and shapes of the connector.

In step (c), a portion of the braided wires is coated with a material suitable for forming a securing element. For example, the portion of the braided wires formed over the tapered/sharpened end of the rod may be coated with polyimide. The polyimide coating may form a ring or cap over the tapered/sharpened end of the rod, such that the ring or cap is not able to slide over a wider portion of the tapered/sharpened end and onto/along the body of the rod.

In step (d), wires buried in the polyimide coat are cut (with precision) with a cutter (e.g., a custom 360° microcutter). This polyimide coating tapered ring structure prevents the plurality of wires (e.g., the braided wires) from being pushed back during insertion into a muscle.

Further, the polyimide coating tapered ring structure enables the braid to be released smoothly from the needle (e.g., when the needle is pulled back). The largest diameter (or analogous feature) of the polyimide coating tapered ring structure is slightly smaller than the needle body diameter.

When certain interaction sites (e.g., recording site, stimulating sites, etc.) are designed to be located at positions along body of wires over the length of the probe (i.e., not just the interaction sites created at the tip of wires by cutting in the step (d)), step (e) can be implemented. Step (e) includes the step of applying another polyimide coating at the tips of the braided plurality of wires exposed by the cutting of step (d). Step (e) includes creating electrical insulations with another polyimide coating at the tip of wires exposed by cutting.

In step (f), interaction sites (e.g., recording sites, stimulating sites, etc.) are disposed on the braided plurality of wires via an ablation process (e.g., a laser ablation process). Step (f) does not need to be carried out if all interaction sites (e.g., recording sites, stimulating sites, etc.) are to be located at the tip of wires cut (e.g., in the cutting step of step (d)). The exposed area of interaction sites (e.g., recording sites, stimulating sites, etc.) by ablation is configurable depending on the application.

The present disclosure describes a multi-electrode probe for use in electromyographic or neural recording and stimulation (e.g., both EMG recording and stimulation; multichannel EMG motor unit potentials (MUPs) and multiple isolated single motor unit (SMU)). The use of highly flexible ultrafine wires in a tubular microbraid according to one or more of the embodiments disclosed herein provides flexible, implantable probes. This add capabilities in electrophysiology, such as allowing chronic recording and stimulation in brains and especially in spinal cords in freely moving animals. Additionally, the microwires are braided in geometries to significantly improve signal reliability through multichannel acquisition, and thereby to gain more MUP and SMU signals per electrode location in electromyography. This ability to obtain several examples of single motor units simultaneously by a single insertion (in contrast to other EMG needles which require multiple insertions with a single recording site, often at the end of the probe), improves signal reliability and reduces pain. The collected data sets can further be suited to machine learning and automated multichannel analysis. Accordingly, the implantable probes of the present disclosure can be used for novel electrodiagnostic monitoring of gradual muscle change in activity after injury or in joint replacement, and therapeutic methods to stimulate, reactivate muscles, manage chronic pain in open-loop or closed-loop systems.

In addition, the implantable probe(s) (e.g., injectable BMEPs) of the present disclosure can be deployed at a target depth in muscle after inserting and removing the center needle. Thus, the implantable probe(s) can be used to give microlevel E-Stim in a narrow local area for optimal E-Stim functions as a therapy, with no additional pain expected due to the very low level of electrical current. Moreover, the high mechanical flexibility and small size of the implantable probe of the present disclosure permits implantation into a muscle for much longer periods of time, facilitating semi-chronic (and/or minimally invasive implanted chronic) E-Stim beyond acute uses. Because the implantable probes (e.g., BMEPs) provide multiple channels which can be used for both E-Stim and EMG recording (e.g., in serial or parallel), these E-Stim designs also allow continuous E-Stim for several weeks in an open-loop system, or adaptive variable E-stim using sensed EMG inputs from the probe in a close-loop system. A portable semichronic EMG recording system with an implantable probe (e.g., BMEP) can be further used as a novel electrodiagnostic tool to monitor muscle changes in activity after disease, surgery or injury during the recovery process. Such applications are currently not available at all in clinics, and they can be only realized with injectable and implantable BMEPs.

Further provided herein is a method of injecting/implanting the probe 100 according one or more of the embodiments disclosed herein. In some embodiments, the method includes positioning the plurality of wires 110 over the core portion 102, penetrating biological tissue with the core portion 102, and withdrawing the core portion 102 while the plurality of wires 110 remain within the biological tissue. An example of this process is illustrated in FIGS. 4A-4C, where the probe 100 is implanted in agar gel so that all elements of the probe 100 can be seen throughout the process. In FIG. 4A, implantable probe 100 (and core needle 102) is illustrated having been inserted into agar gel 160 (e.g., 0.5% and 5% agar gels). Agar gel 160 act as a material emulating artificial neural tissue and muscles, and allowing visualization of the processes. In FIG. 4B, the core portion 102 is illustrated being released (e.g., removed) from agar gel 160. Implantable probe 100 remains placed in agar gel 160 (e.g., representing biological material). FIG. 4C illustrates, a close up of implantable probe 100 while the core portion 102 is being removed.

Referring now to FIGS. 5A-5D, a block diagram of implantable probe 100 being used in connection with a biological material 126 (e.g., a muscle) is illustrated. In FIG. 5A, the implantable probe 100, with core portion 102, is positioned just outside of biological material 126 (e.g., skin, muscle, etc.) prior to being inserted. In FIG. 5B, after being inserted, a plurality of interaction sites (e.g., recording sites) make contact with the biological material 126 (e.g., a muscle) such that a plurality of electrical signals (e.g., MUPs) of biological material 126 can be measured. Such a use of implantable probe 100 offers the advantage of being able to take multiple measurements (e.g., of MUPs) from multiple locations (e.g., serially or simultaneously) without moving the tip of a needle around to take multiple measurements, which would cause some negative consequences (e.g., pain, discomfort, greater risk of injury or damage to the biological material, etc.). In certain embodiments, the multichannel analysis or measurement is done simultaneously. In FIG. 5B, as the core portion 102 is withdrawn, the securing element 150 ‘unjams’ or releases from the core portion 102, allowing the braid of implantable probe 100 to remain in the biological material 126. In FIG. 5C, the needle is fully removed from the plurality of wires 110, leaving only the probe 100 implanted in the biological material 126 (FIG. 5D).

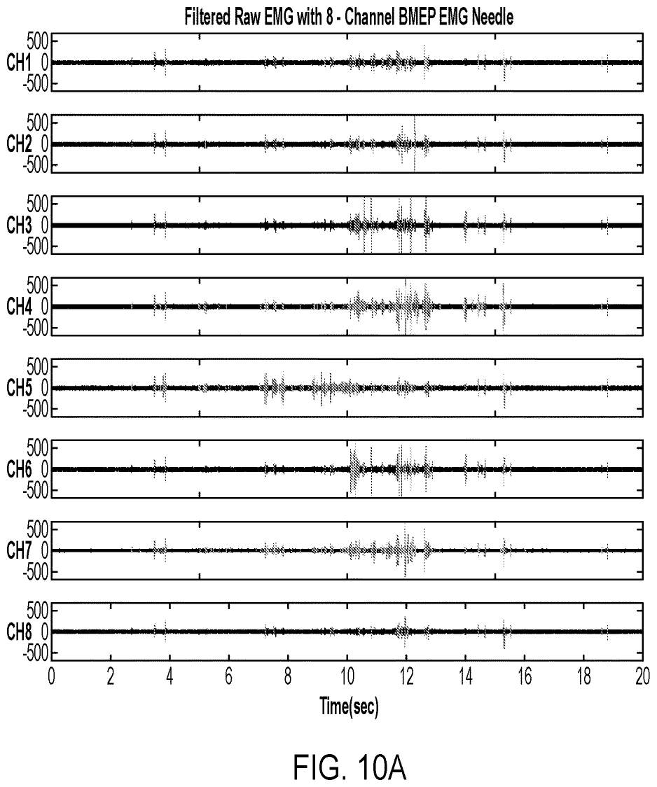

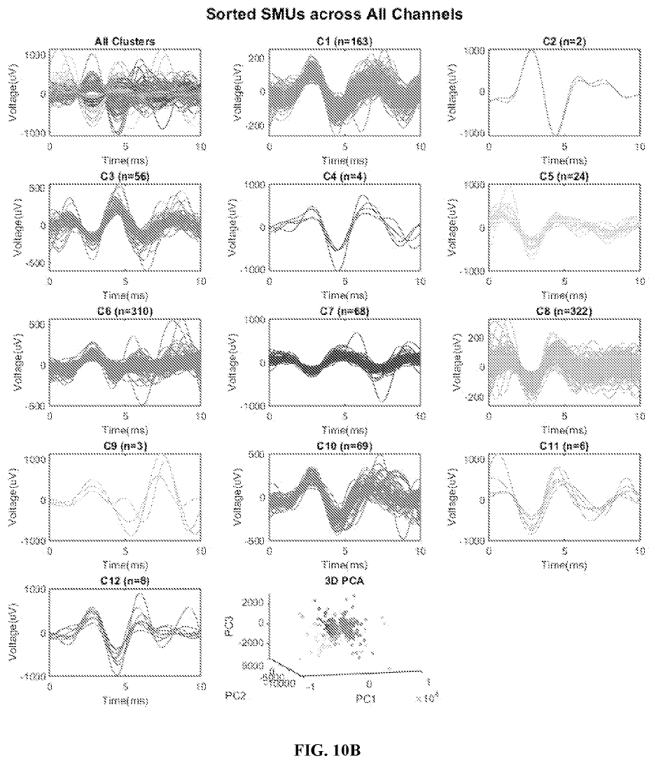

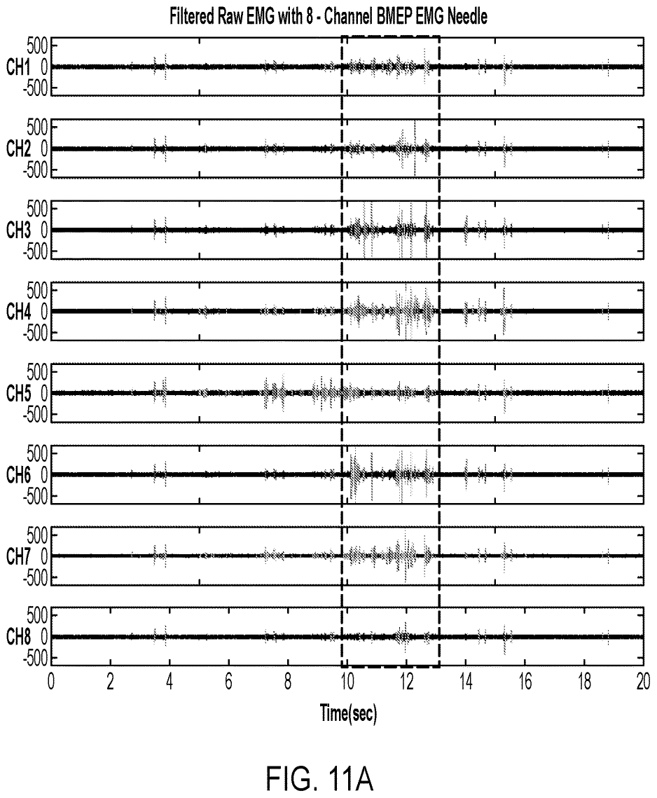

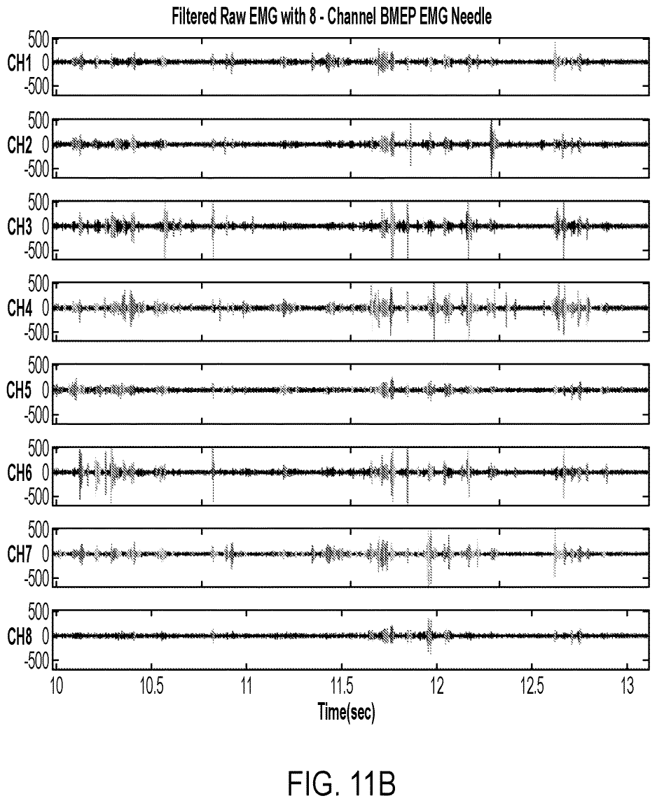

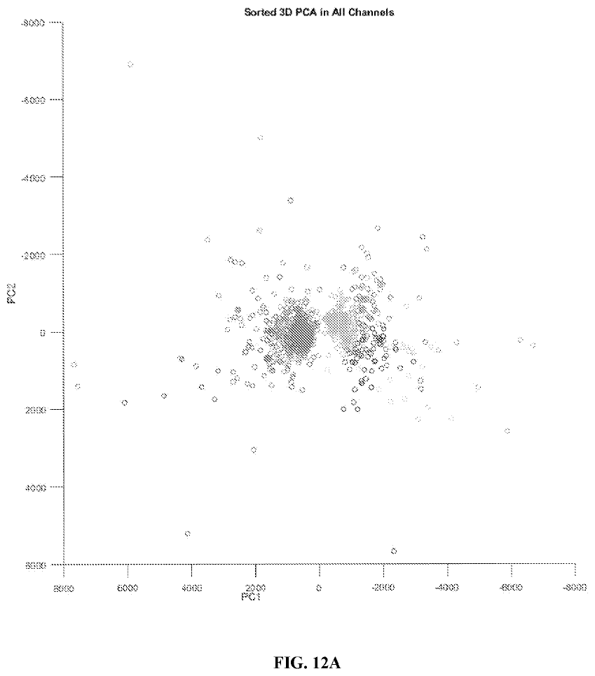

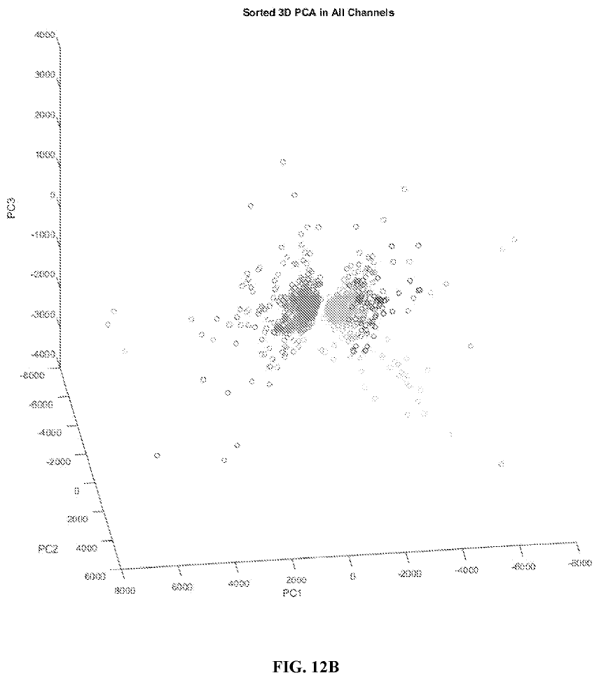

Turning to FIG. 9, an example of a fully assembled deployment and connection mechanism is shown during EMG recording in a rat. Data collected from the EMG recording in a rat is shown in FIGS. 10A-12B.

### Equivalents

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. An implantable probe, comprising:
a plurality of wires configured for at least one of electromyography and electrical stimulation; and
a securing element formed at an end of the plurality of wires;
wherein the plurality of wires are in a braided configuration; and
wherein the plurality of wires are configured to transmit multiple electrical signals in connection with a biological material.

2. The implantable probe of claim 1, further comprising a removable core portion.

3. The implantable probe of claim 2, wherein the plurality of wires are configured to be braided around an exterior surface of the core portion.

4. The implantable probe of claim 2, wherein the securing element is configured to maintain a position of the plurality of wires with respect to the core portion during implantation of the probe.

5. The implantable probe of claim 4, wherein the securing element is configured to release from the core portion when the core portion is withdrawn from an implantation site.

6. The implantable probe of claim 1, wherein the securing element includes a ring or cap.

7. The implantable probe of claim 1, wherein the securing element comprises a polyimide material.

8. The implantable probe of claim 1, wherein the plurality of wires are highly flexible multichannel microelectrodes.

9. The implantable probe of claim 1, wherein the plurality of wires are tubularly braided microwires.

10. The implantable probe of claim 1, wherein the plurality of wires are configured to release from a core portion after insertion of the implantable probe into a biological material.

11. The implantable probe of claim 10, wherein the plurality of wires include a tapered end configured to release from a core portion upon withdrawal of the core portion from the biological material.

12. A syringe-based implantable probe system comprising:
(i) a syringe;
(ii) a core portion attached to a plunger within the syringe; and
(iii) the implantable probe of claim 1 arranged and disposed to be positioned over the core portion.

13. The system of claim 12, wherein the core portion includes a PTFE or polyimide coating.

14. A method of making the implantable probe of claim 1, the method comprising the steps of:
(a) tubularly braiding the plurality of wires on a core portion;
(b) making electrical connections between the braided plurality of wires on the core portion and respective channels of a multichannel connector;
(c) applying a coating on a portion of the braided microwires; and
(d) cutting the braided plurality of wires within the coating to form a coating tapered ring structure.

15. The method of claim 14, further comprising the step of:
(e) applying another polyimide coating at the tips of the braided plurality of wires exposed by the cutting of step (d).

16. The method of claim 15, further comprising the step of:
(f) disposing a plurality of interaction sites on the braided plurality of wires via an ablation process.

17. The method of claim 14, wherein the braided plurality of wires are in a single 12 filament tubular braid configuration.

18. The method of claim 14, wherein the braided plurality of wires are in a double 12 filament tubular braid configuration.

19. The method of claim 14, wherein during step (b) the connector is attached to the syringe body by at least one of a mechanical holder and a magnetic holder.

20. The method of claim 14, wherein the coating is applied over only the portion of the braided microwires corresponding to a distal end of the core portion.

21. The method of claim 14, wherein the coating tapered ring portion is configured to maintain a position of the tubularly braided plurality of wires with respect to the core portion during insertion of the probe into a muscle.

22. The method of claim 21, wherein the coating tapered ring structure is further configured to release from the core portion when the core portion is withdrawn from the muscle.
